(19) United States
(12) Patent Application Publication
(10) Pub. No.: US 2021/0093887 A1
(43) Pub. Date: Apr. 1, 2021

(54) MULTI-MODULE THERAPEUTIC DEVICE FOR SKINCARE

(71) Applicant: LIGHT TREE, Amstelveen (NL)

(72) Inventors: ALAIN DIJKSTRA, Amstelveen (NL); YVONNE JOHANNA MARGARETHA HOUTHUIJS, Amstelveen (NL)

(73) Assignee: LIGHT TREE, Amstelveen (NL)

(21) Appl. No.: 16/996,907

(22) Filed: Aug. 19, 2020

(30) Foreign Application Priority Data
Sep. 30, 2019 (IN) 201911039456

(51) Int. Cl. A61N 5/06

(52) U.S. Cl. CPC A61N 5/0616; A61N 2005/0644; A61N 2005/0663; A61N 2005/0659; A61N 2005/0651

(57) ABSTRACT

A multi-module therapeutic device for skincare comprises a housing capable of being ergonomically held in one hand, wherein a bottom surface of the housing is planar horizontal in shape, a plurality of Light Emitting Diodes (LEDs) configured to provide light therapy through the emission of electromagnetic radiation, a vibration motor configured to facilitate a provision of massage therapy, a metal sheet enclosed within the housing including a plurality of LED apertures for receiving the plurality of LEDs, wherein the metal sheet is configured to act as a medium for the application of massage therapy and thermotherapy, a medicament dispenser configured to dispense a medicament on actuation, and a control panel provided along a top surface of the housing, wherein the control panel is operably connected with the plurality of LEDs, the vibration motor, and the medicament dispenser.

### TECHNICAL FIELD

The present invention relates generally to therapeutic devices. More specifically, but not exclusively, the present invention relates to therapeutic devices that include multiple distinct therapeutic modules that are capable of providing multiple kinds of therapies through a single device, for treating skin related ailments in human beings.

### BACKGROUND ART

Human skin is the largest organ in a human body, and provides an outer covering to the human body, thereby protecting internal tissues and organs against infection, loss of water in the form of dehydration, and harmful radiations including ultraviolet light and the like. Also, the skin is capable of generating Vitamin D on the absorption of sunlight. Therefore, proper care of the skin is required for the maintenance of good health. External environmental agents and internal body conditions may affect the skin resulting in skin-related disorders or altered skin conditions like wrinkles, sun spots, acne, discoloration, scars, pigmentation, rosacea, actinic keratosis, and unwanted hair growth to name a few. As the characteristics of the skin are also a measure of the aesthetic appeal of individuals, most individuals prefer non-invasive or minimally invasive therapies for the treatment of skin ailments.

With the advancements in Research and Development (R&D) pertinent to therapeutic devices, several novel kinds of non-invasive or minimally invasive therapies have been suggested for the treatment of human skin related diseases and disorders. For example, in light therapy, also known as Low Level Laser Therapy (LLLT), Photo-Dynamic Therapy (PDT) or Photo-Bio-Modulation (PBM), low-intensity lasers and Light Emitting Diodes (LEDs) are used to generate irradiation patterns of desired characteristics. Further, there is an electrical therapy wherein electrical stimulation is used for treating the skin ailments. Here again, low magnitude electric currents are passed through the skin to produce several therapeutic effects, including, but not limited to, toning of muscles in the body and micro-lifting of the face. In vibrational massaging of the skin, mechanical vibrations are used to improve skin conditions and also to reduce stress. However, for using each of the aforementioned treatment methods, there arises the requirement for configuring a dedicated device corresponding to each one of the individual treatment methods.

It has further been observed that while one kind of treatment may be compatible with certain individuals owing to their physiological characteristics such as age, sex, skin type, and pre-existing conditions, etc., the same treatment may not be compatible with another group of individuals with a different set of physiological characteristics. Further, the efficacy of the kind of treatment may also vary as per the variations in the kind of ailments being addressed. For example, while LLLT may be effective in the treatment of acne in Latino individuals, the electromagnetic radiation may not be able to assist as much in pain relief as electrical therapy. Vice versa may be true of Caucasian individuals. Therefore, it might become desirable or rather necessary, to purchase several such devices to address the issues that arise out variations in physiological characteristics and kind of ailments, forcing an individual or a treatment facility to incur excessive expenditure.

Therefore there is a requirement in the art for a multi-module therapeutic device that does not suffer from the aforementioned deficiencies.

### OBJECTS OF THE INVENTION

Some of the objects of the present invention are as stated below:

An object of the present invention is to provide a personal skincare device that includes several therapeutic modules including light therapy module, massage module, medicament module, thermotherapy module for the treatment of a human skin disorder;

Another object of the present invention is to provide a personal skincare device that may be ergonomically held in the hand of a user;

Yet another object of the present invention is to provide a personal skincare device comprising Surface Mount Technology (SMT) based Light Emitting Diodes (LEDs) for the light therapy module;

Yet another object of the present invention is to provide a personal skincare device that uses a lightweight metal sheet for providing thermotherapy and massage therapy;

An object of the present invention is to provide a personal skincare device which can be operated using a remote communication device;

Also, an object of the present invention is to provide a personal skincare device configured for wireless charging of a power supply unit included in the device.

Other objects, aspects, features, and goals of the present invention will be better understood from the following detailed description.

### SUMMARY

According to a first aspect of the present invention, there is provided a multi-module therapeutic device for skincare, the multi-module therapeutic device comprising a housing capable of being ergonomically held in one hand, wherein a bottom surface of the housing is planar horizontal in shape, a plurality of Light Emitting Diodes (LEDs) configured to provide light therapy through emission of electromagnetic radiation, a vibration motor configured to facilitate a provision of massage therapy, a metal sheet enclosed within the housing including a plurality of LED apertures for receiving the plurality of LEDs, wherein the metal sheet is configured to act as a medium for application of massage therapy and thermotherapy, a medicament dispenser configured to dispense a medicament on actuation, and a control panel provided along a top surface of the housing, wherein the control panel is operably connected with the plurality of LEDs, the vibration motor, and the medicament dispenser.

In one embodiment of the invention, the metal sheet is configured to be heated from heat energy produced by the plurality of LEDs.

In one embodiment of the invention, the multi-module therapeutic device further comprises an electrical heating element in thermal communication with the metal sheet.

In one embodiment of the invention, the metal sheet is configured to be heated from heat energy received from one or more of the plurality of LEDs and the electrical heating element.

In one embodiment of the invention, the control panel includes at least a first button configured to actuate the plurality of LEDs, a second button configured to actuate the vibration motor, and a third button configured to actuate the medicament dispenser.

In one embodiment of the invention, the housing is in a shape of a computer mouse.

In one embodiment of the invention, the plurality of LEDs is configured to emit electromagnetic radiation in red and infrared wavelength ranges of the electromagnetic spectrum.

In one embodiment of the invention, the plurality of LEDs has been provided with a plurality of respective auxiliary optics configured to adjust a beam angle of the electromagnetic radiation emitted by the plurality of LEDs.

In one embodiment of the invention, the plurality of auxiliary optics includes a plurality of convex lenses.

In one embodiment of the invention, the vibration motor is an eccentric mass motor and is coupled with the metal sheet for providing massage therapy.

In one embodiment of the invention, the metal sheet is made out of aluminum.

In one embodiment of the invention, the medicament dispenser is configured to be actuated by an intermediary piston mechanism comprising a piston head and a piston shaft, a compression plate provided at a bottom of the piston shaft, wherein the compression plate includes a plurality of medicament apertures and a plurality of compression springs provided between a bottom surface of the compression plate and a top surface of a channel plate, wherein a bottom surface of the channel plate includes a plurality of medicament channels that are co-axial with the plurality of medicament apertures.

In one embodiment of the invention, the metal sheet further includes a plurality of medicament dispensing apertures that are coaxial with the plurality of medicament apertures and the plurality of medicament channels.

In one embodiment of the invention, a third button of the control panel is in a form of a scroll-wheel capable of rotating about an axis, wherein the scroll-wheel includes a plurality of protrusions adapted to be in contact with the intermediary piston mechanism on the rotation of the scroll-wheel.

In one embodiment of the invention, the medicament dispenser includes a medicament reservoir.

In one embodiment of the invention, the control panel further includes an irradiation control interface, a medicament flow control interface, a vibration control interface, and a temperature control interface.

In one embodiment of the invention, the irradiation control interface is configured to control emission characteristics of the electromagnetic radiation emitted by the plurality of LEDs, the medicament flow control interface is configured to control a flow rate of the medicament from the medicament dispenser, the vibration control interface is configured to control vibration characteristics of the vibration of the metal sheet and the temperature control interface is configured to control a temperature of the metal sheet.

In one embodiment of the invention, the irradiation control interface, the medicament flow control interface, the vibration control interface, and the temperature control interface are configured to be actuated through a wired or wireless connection with a remote communication device.

In one embodiment of the invention, the multi-module therapeutic device further comprises a processor, a memory unit and a communication interface, wherein the memory unit includes machine-readable instructions that when executed by the processor, enable the processor to receive an actuation input from the control panel to actuate one or more of the vibration motor and the plurality of LEDs, receive one or more of an irradiation control signal, a medicament flow control signal, a vibration control signal and a temperature control signal and perform one or more of modifying characteristics of the irradiation emitted by the plurality of LEDs, modifying a flow rate of the medicament from the medicament dispenser, modifying the vibration characteristics of the vibration of the metal sheet, and modifying the temperature of the metal sheet.

According to a second aspect of the present invention, there is provided a method for providing multi-module therapy, the method comprising steps of providing a multi-module therapeutic device for skincare, the multi-module therapeutic device comprising a housing capable of being ergonomically held in one hand, wherein a bottom surface of the housing is planar horizontal in shape, a plurality of Light Emitting Diodes (LEDs) configured to provide light therapy, a vibration motor configured to facilitate the provision of massage therapy, a metal sheet enclosed within the housing including a plurality of LED apertures for receiving the plurality of LEDs, wherein the metal sheet is further configured to act as a medium for the application of the massage therapy and thermotherapy, a medicament dispenser, configured to dispense a medicament on actuation, and a control panel provided along a top surface of the housing, wherein the control panel is operably connected with the plurality of LEDs, the vibration motor, and the medicament dispenser, receiving an actuation input from the control panel to actuate one or more of the vibration motor and the plurality of LEDs, receiving one or more of an irradiation control signal, a medicament flow control signal, a vibration control signal and a temperature control signal and performing one or more of modifying characteristics of the irradiation emitted by the plurality of LEDs, modifying the flow rate of the medicament from the medicament dispenser, modifying the vibration characteristics of the vibration of the metal sheet, and modifying the temperature of the metal sheet.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelength, wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of this specification, the term “medicament” includes, within its scope, facial serums, skin moisturizers, oils, and pharmaceutical compositions, etc. that may be available in form of liquids, gels, emulsions, and solutions, etc. and are used for treating skin-related conditions and/or enhancing overall aesthetic and general health of the skin of a user.

### BRIEF DESCRIPTION OF ACCOMPANYING DRAWINGS

So that the manner in which the above-recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1A illustrates a top view of a multi-module therapeutic device for skincare, in accordance with an embodiment of the present invention;

FIG. 1B illustrates an exploded view of the multi-module therapeutic device of FIG. 1A;

FIG. 1C illustrates a sectional view of the multi-module therapeutic device of FIG. 1A;

FIG. 1D illustrates a bottom view of the multi-module therapeutic device of FIG. 1A;

FIG. 1E illustrates an enlarged view of an LED aperture of the multi-module therapeutic device of FIG. 1A;

FIG. 2 illustrates a logical diagram of the multi-module therapeutic device, in accordance with an embodiment of the present invention

FIG. 3A illustrates a multi-planar sectional view of the multi-module therapeutic device of FIG. 1A, with a compression plate in a rest position;

FIG. 3B illustrates a multi-planar sectional view of the multi-module therapeutic device of FIG. 1A, with the compression plate in a compressed position;

FIG. 4 illustrates wireless charging of the multi-module therapeutic device of FIG. 1A, in accordance with an embodiment of the present invention; and

FIG. 5 illustrates connection of the multi-module therapeutic device with a remote communication device through a shared network, in accordance with an embodiment of the present invention.

### DETAILED DESCRIPTION

While the present invention is described herein by way of example using embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claims. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned.

Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes. Any discussion of documents acts, materials, devices, articles, and the like is included in the specification solely to provide a context for the present invention. It is not suggested or represented that any or all of these matters form part of the prior art base or were common general knowledge in the field relevant to the present invention. In this disclosure, whenever a composition or an element or a group of elements is preceded with the transitional phrase “comprising”, it is understood that we also contemplate the same composition, element or group of elements with transitional phrases “consisting of, “consisting”, “selected from the group of consisting of, “including”, or “is” preceding the recitation of the composition, element or group of elements and vice versa.

The present invention is described hereinafter by various embodiments with reference to the accompanying drawings, wherein reference numerals used in the accompanying drawing correspond to the like elements throughout the description. This invention may, however, be embodied in many different forms and should not be construed as limited to the embodiment set forth herein. Rather, the embodiment is provided so that this disclosure will be thorough and complete and will fully convey the scope of the invention to those skilled in the art. In the following detailed description, numeric values and ranges are provided for various aspects of the implementations described. These values and ranges are to be treated as examples only and are not intended to limit the scope of the claims. In addition, a number of materials are identified as suitable for various facets of the implementations. These materials are to be treated as exemplary and are not intended to limit the scope of the invention.

It is envisaged here that a multi-module therapeutic device (hereinafter also referred to as “the device”) for skincare be provided that may be portable and capable of being ergonomically held in hands of the user. In that regard, the device may be given a form resembling a computer mouse as it is a widely used article and its utilization would be rather intuitive for a user. The device is further envisaged to include several modules for several different kinds of therapies such as light therapy (also known as Low Level Laser Therapy (LLLT), Photo-Dynamic Therapy (PDT) or Photo-Bio-Modulation (PBM)), vibrational massage therapy, thermotherapy and application of a medicament on affected areas of the skin. Each one of the modules is envisaged to be able to work independently and in tandem with each other thereby providing enhanced therapeutic benefits. It is further envisaged that activation and deactivation of the several modules be possible using easily accessible buttons on a control panel of the device, wherein additional control interfaces may be provided for regulation of the operational characteristics of the several different therapeutic modules.

The device is also envisaged to include capabilities for connecting with a remote communication device for receiving control signals and be able to be charged, through both wired and wireless connections. It is further envisaged that the device is simple in construction and be made of lightweight materials for the convenience of the users using the device. Referring to the figures now, the invention will be described in more detail.

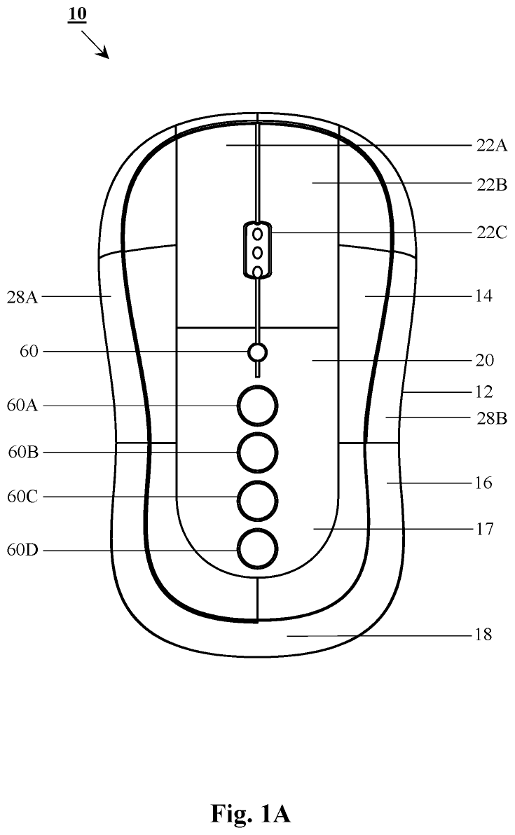

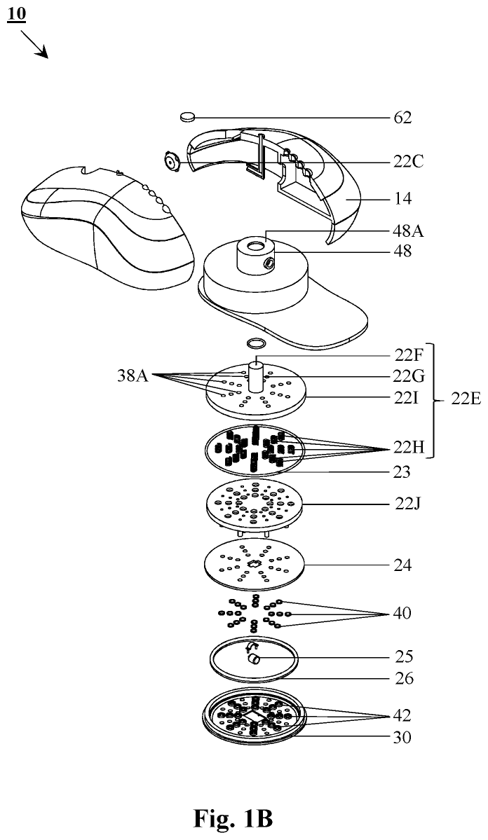

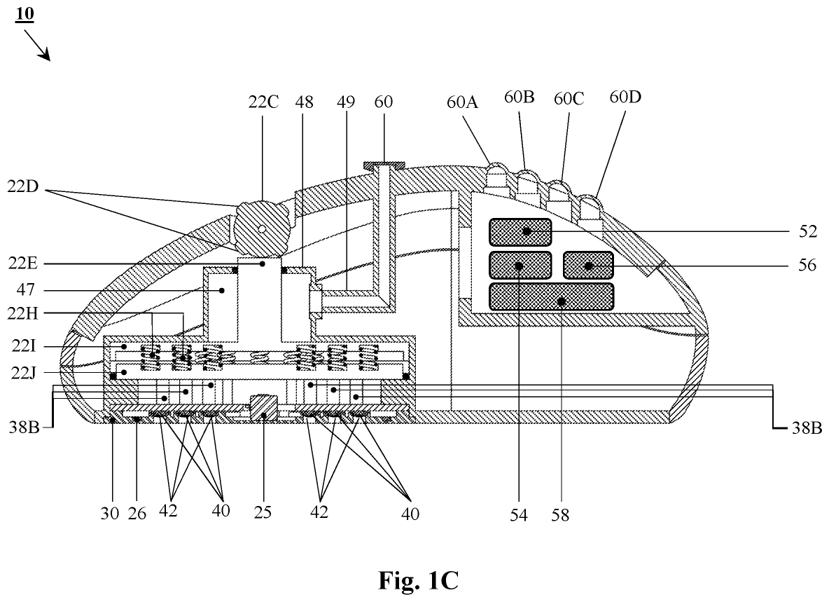

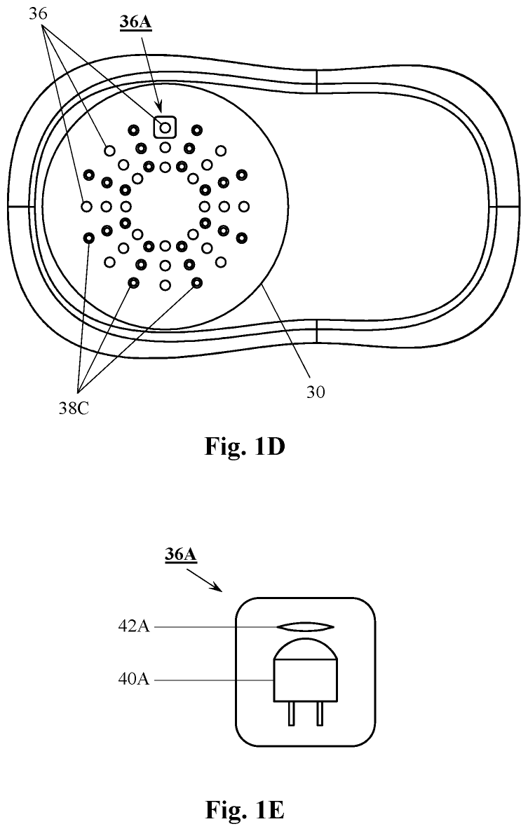

FIG. 1A illustrates a top view of a multi-module therapeutic device 10 (hereinafter also referred to as “the device 10”) for skincare, in accordance with an embodiment of the present invention. The device 10 includes a housing 14 capable of being held ergonomically in one hand. In several embodiments of the present invention, the housing 14 is in the shape of a computer mouse. Moreover, a bottom surface 12 of the housing 14 is planar horizontal in shape. The planar horizontal shape of the bottom surface 12 facilitates a convenient translational motion of the device 10 over the skin surface of the user. Further, the housing 14 consists of a wall 16 projected upward towards a top surface 17 of the housing 14 from the bottom surface 12. The wall 16 contours a sidewall structure 18 surrounding the housing 14. There is also provided is a control panel 20 provided along the top surface 17 and enclosed within the housing 14. The control panel 20 includes a first button 22A, a second button 22B, and a third button 22C. The respective functions of the first 22A, the second 22B, and the third 22C buttons will be elucidated in the following description. Also, the control panel 20 includes an inlet 60 for receiving a medicament, an irradiation control interface 60A, a medicament flow control interface 60D, a vibration control interface 60B, and a temperature control interface 60C, each of which will be elucidated upon in detail, in the following discussion.

One portion 28A of the sidewall structure 18 encloses the control panel 20 on one side, is adapted to provide support for a thumb of the user, assuming that the user is right-handed, and another portion 28B of the sidewall structure 18 enclosing the control panel 20 on the other side, is adapted to provide the support for the fingers of the user. While the reverse may be true in case the user opts to use the device 10 with their left hand. The housing 14 with the control panel 20 forms a three-dimensional closed curve configuration of the device 10, making the device 10 ergonomic to use.

FIG. 1B illustrates an exploded view of the multi-module therapeutic device 10 of FIG. 1A. As depicted in FIG. 1B, the housing 14 is a split type housing and can be injection molded from several polymeric materials such as Polypropylene, Low-Density Poly Ethylene (LDPE), High-Density Poly Ethylene (HDPE), Polycarbonate, Poly Vinyl Chloride (PVC) and the like. There is also provided a cap 62 for the inlet 60, to prevent the ingress of external agents such as dust and moisture, into the device 10. It has also been depicted in FIG. 1B that the third button 22C of the control panel 20 is in the form of a scroll-wheel capable of rotating about an axis. There is also provided a medicament dispenser 48, with a top-end 48A of the medicament dispenser 48 being capable of making contact with the scroll-wheel shaped third button 22C. The medicament dispenser 48 is configured to dispense a medicament on actuation.

For actuation of the medicament dispenser 48, the device 10 includes an intermediary piston mechanism 22E. The intermediary piston mechanism 22E includes a piston head 22F and a piston shaft 22G. The piston head 22F and the piston shaft 22G may be made up of rigid polymeric materials, lightweight metals such as aluminum and magnesium, or metal composites, etc. Further, the intermediary piston mechanism 22E includes a compression plate 22I provided at the bottom end of the piston shaft 22G. In several embodiments, the piston head 22F, the piston shaft 22G, and the compression plate 22I may be integrated to form a single functional unit. Integration of the aforementioned components allows them to be injection molded or die-casted in a single manufacturing step thereby reducing the cost of tooling, production, and assembly, had the piston head 22F, the piston shaft 22G, and the compression plate 22I were separately fabricated. Further, the compression plate 22I includes a plurality of medicament apertures 38A. The intermediary piston mechanism 22E further includes a channel plate 22J. A plurality of compression springs 22H has been provided between a bottom surface of the compression plate 22I and a top surface of the channel plate 22J. Also there has been provided a sealing ring 23 configured to provide a leak proof sealing between the compression plate 22I and the channel plate 22J. The sealing ring 23 may be made up of chemical resistant rubber such as Neoprene.

Further, as illustrated in FIG. 1B, the device 100 includes a Printed Circuit Board (PCB) 24 for mounting a plurality of LEDs 40. The plurality of LEDs 40 is configured to provide light therapy through the emission of electromagnetic radiation. In that regard, in several embodiments, the plurality of LEDs 40 may be configured to emit electromagnetic radiation in red and infrared wavelength ranges of the electromagnetic spectrum. However, other therapeutic wavelengths are envisaged to be within the scope of the invention. The LEDs are characterized by their superior power efficiencies, smaller sizes, rapidity in interfacing, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high power LEDs, etc. Materials used in the one or more LEDs may vary from one embodiment to another depending upon the frequency of radiation required. Different frequencies can be obtained from LEDs made from pure or doped semiconductor materials.

Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultra Violet (UV) LEDs may be combined with europium based phosphors to generate red and blue lights and copper and zinc doped zinc sulfide-based phosphor to generate green light.

The plurality of LEDs 40 may be installed with the PCB 24 through Surface Mount Technology (SMT). SMT permits the creation of smaller PCB designs by allowing components to be placed closer together on the board that makes the device more lightweight and compact. The SMT process is faster to set up for production and requires less manufacturing cost than its counterpart, through-hole technology because it does not require the circuit board to be drilled for assembly. In several alternate embodiments, the plurality of LEDs 40 may be installed with the PCB 24 via through-hole technology. In several embodiments, the assembly of the plurality of LEDs 40 and the PCB 24 may be embodied as flexible LED panels such as Organic LED (OLED) based flexible panels or inorganic LED-based flexible panels.

Such OLED panels may be generated by depositing organic semiconducting materials over Thin Film Transistor (TFT) based substrates. Further, discussion on generation of OLED panels can be found in Bardsley, J. N. (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557B2, titled “Roll-to-roll fabricated light sheet and encapsulated semiconductor circuit devices”, which is included herein in its entirety, by reference. In several embodiments, the plurality of LEDs 40 may also be micro-LEDs described through U.S. Pat. Nos. 8,809,126B2, 8,846,457B2, 8,852,467B2, 8,415,879B2, 8,877,101B2, 9,018,833B2, and their respective family members, assigned to NthDegree Technologies Worldwide Inc., which are included herein by reference, in their entirety. The plurality of LEDs 40, in that regard, may be provided as a printable composition of the micro-LEDs, printed on the PCB 24 acting as a substrate.

In several embodiments, the plurality of LEDs 40 has been provided with a plurality of respective auxiliary optics 42 configured to adjust beam angle of the electromagnetic radiation emitted by the plurality of LEDs 40. The plurality of auxiliary optics 42 may assist in controlling other emission characteristics of the emitted electromagnetic radiation, such as point of incidence, angle of incidence, intensity, and wavelengths through the interference of two or more beams. In that regard, the plurality of auxiliary optics 42 may include convex lenses, concave lenses, Total Internal Reflection (TIR) lenses, and the like. Further, the housing 14 encloses a vibration motor 25 configured to facilitate a provision of massage therapy. In several embodiments of the invention, the vibration motor 25 is an eccentric mass motor and is coupled with a metal sheet 30 for providing massage therapy. In several alternate embodiments, the vibration motor 25 may be a piezo-electric material based motor. The metal sheet 30 is envisaged to be made up of a lightweight material such as aluminum owing to its superior thermal conductivity and vibration transmissibility. Therefore, the metal sheet 30 is configured to act as a medium for the application of massage therapy and thermotherapy.

In several embodiments, the metal sheet 30 is configured to be heated from heat energy produced by the plurality of LEDs 40. In several alternate embodiments, like the one depicted in FIG. 1B, an electrical heating element 26 may be provided within the housing 14. In such embodiments, the metal sheet 30 may be configured to be heated from heat energy received from one or more of the plurality of LEDs 40 and the electrical heating element 26. The control panel 20 is operably connected to the plurality of LEDs 40, the vibration motor 25, and the medicament dispenser 48. More specifically, the first button 22A is configured to actuate the plurality of LEDs 40, the second button 22B is configured to actuate the vibration motor 25 and the third button 22C is configured to actuate the medicament dispenser 48.

FIG. 1C illustrates a sectional view of the multi-module therapeutic device 10 of FIG. 1A. As depicted in FIG. 1C, the medicament dispenser 48 includes a medicament reservoir 47 configured to store the medicament. The medicament reservoir 47 can be supplied with the medicament through the inlet 60 and via a filling channel 49. Also, a bottom surface of the channel plate 22J includes a plurality of medicament channels 38B that is coaxial with the plurality of medicament apertures 38A of the compression plate 22I. Further, FIG. 1C illustrates the scroll-wheel construction of the third button 22C. Also depicted in FIG. 1C is that the third button 22C includes a plurality of protrusions 22D that are spaced apart along the circumference of the scroll-wheel design. The plurality of protrusions 22D are configured to come in contact with, and thereby apply downward pressure on the intermediary piston mechanism 22E, on the rotation of the scroll-wheel.

Also, the electrical heating element 26 is in thermal communication with the metal sheet 30, and the vibration motor 25 is mechanically coupled with the metal sheet 30. Also, the device 10 includes a processor 52, a memory unit 54, a communication interface 56, and a power supply unit 58. The processor 52 may be a general-purpose processor, a Field Programmable Gate Array (FPGA) or an Application Specific Integrated Circuit (ASIC), etc. Additionally, the memory unit 54 may be a volatile memory unit such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

The communication interface 56 is configured to allow the device 10 with other communication devices through a shared network. In that regard, the communication interface 56 may include ports such as Universal Serial Bus (USB) port, Video Graphics Array (VGA) port, and High Definition Multimedia Interface (HDMI) port for wired connections. The communication interface 56 may also include modems for wireless communication, such as a Wireless Fidelity (Wi-Fi) modem for data transfer or a GSM modem for mobile telephony, and GPRS modems for connecting to the Internet or other Wide Area Networks through protocols standardized by 3GPP. The power supply unit 58 may be a rechargeable or a replaceable battery. The rechargeable batteries may be made out of Lithium-Ion or Lithium-Polymer based technologies, Nickel Metal Hydride batteries and the like, that may be capable of being charged through wired or wireless means.

FIG. 1D illustrates a bottom view of the multi-module therapeutic device 10 of FIG. 1A. The metal sheet 30 further includes a plurality of medicament dispensing apertures 38C. The medicament dispensing apertures 38C that are coaxial with the plurality of medicament apertures 38A and the plurality of medicament channels 38B, thus providing a route for dispensing the medicament during usage of the device 10. The metal sheet 30 also includes a plurality of LED apertures 36 for receiving the plurality of LEDs 40. The plurality of respective auxiliary optics 42 may also be received in the plurality of LED apertures 36. FIG. 1E illustrates an enlarged view of an LED aperture 36A of the multi-module therapeutic device 10 of FIG. 1A.

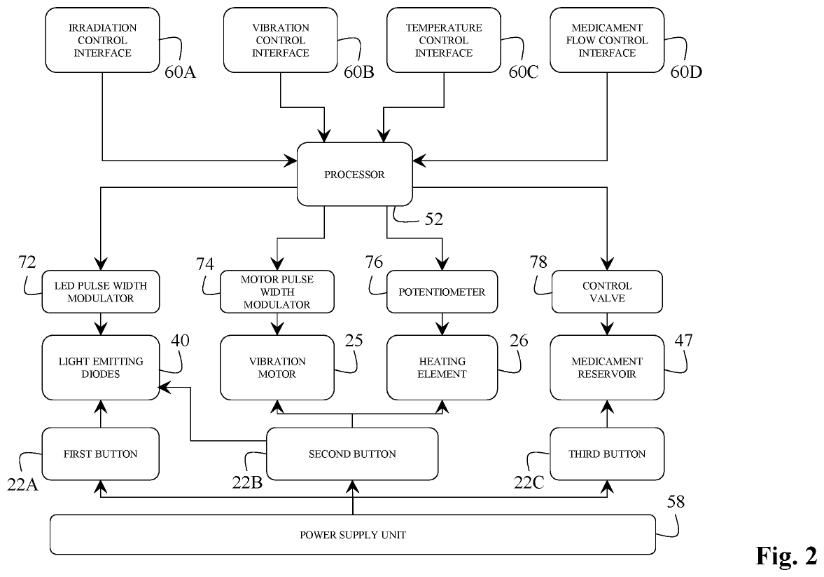

FIG. 2 illustrates a logical diagram of the multi-module therapeutic device 10, in accordance with an embodiment of the present invention. FIG. 2 illustrates the irradiation control interface 60A, the vibration control interface 60B, the temperature control interface 60C, and the medicament flow control interface 60D connected with the processor 52. The irradiation control interface 60A is configured to control emission characteristics of the electromagnetic radiation emitted by the plurality of LEDs 40. Further, the medicament flow control interface 60D is configured to control a flow rate of the medicament from the medicament dispenser 48. Similarly, the vibration control interface 60B is configured to control vibration characteristics of the vibration of the metal sheet 30 by controlling the motor characteristics of the vibration motor 25. Also, the temperature control interface 60C is configured to control the temperature of the metal sheet 30 by controlling one or more of the emission characteristics of the irradiation emitted by the plurality of LEDs 40 and the current being supplied to the electrical heating element 26.

Further, it can be seen from FIG. 2, that the first button 22A, the second button 22B, and the third button 22C are operably connected with the power supply unit 58. In that regard, the first button 22A may act as a power supply switch for the plurality of LEDs 40, to enable and disable light therapy. Similarly, the second button 22B may act as a power supply switch for the plurality of LEDs 40 and/or the electrical heating element 26, to enable and disable thermotherapy, and for the vibration motor 25, to enable and disable vibration massage therapy. Also, the third button 22C may act as an actuator and a power supply switch, to enable and disable actuation of the medicament dispenser 48, and thus to enable and disable dispensing of the medicament. However, based on the settings of the irradiation control interface 60A, the vibration control interface 60B, the temperature control interface 60C, and the medicament flow control interface 60D, the processor 52 may regulate the characteristics of the corresponding therapies.

For example, the processor 52 may regulate emission characteristics such as intensity and pulse width of the electromagnetic radiation by deploying an LED pulse width modulator 72. Further, the processor 52 may regulate the characteristics of thermotherapy, such as temperature and rate of heat transfer, etc. by deploying the LED pulse width modulator 72 and/or the potentiometer 76. Similarly, the processor 52 may regulate the characteristics of vibration massage therapy, such as amplitude and frequency of vibrations, by deploying a motor pulse width modulator 74. Also, the processor 52 may control the flow rate of the medicament from the medicament reservoir 47 by deploying a control valve 78. However, a person skilled in the art would appreciate that there are several alternative open-loop and closed-loop control strategies that may be deployed to regulate characteristics of the aforementioned therapies without departing from the scope of the present invention. The following description elucidates the actuation of the medicament dispenser 48 for dispensing the medicament to the skin of the user.

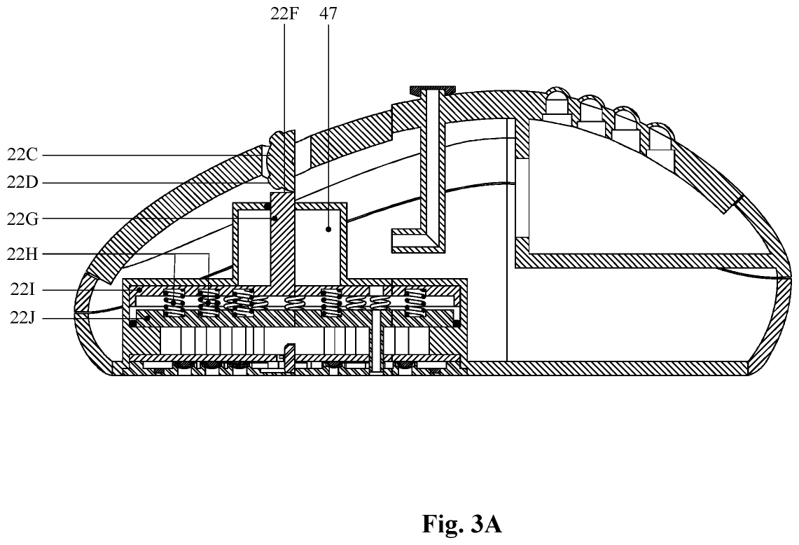

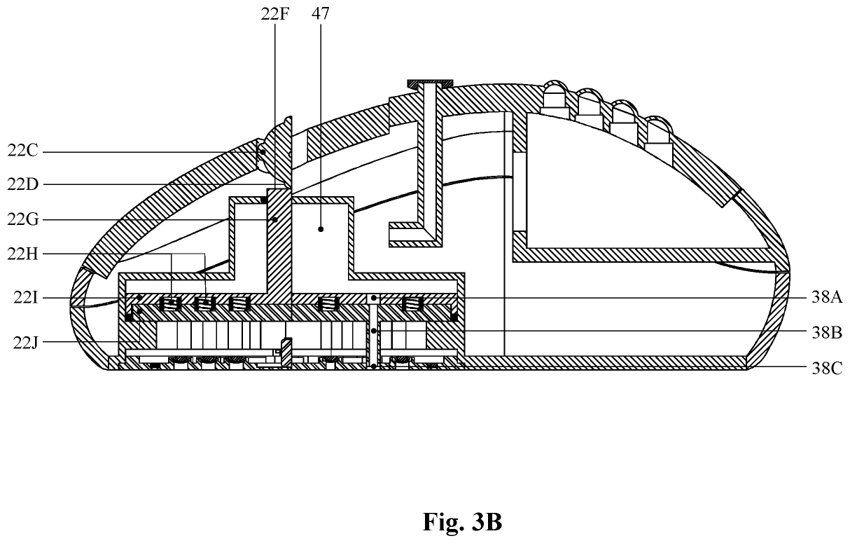

FIG. 3A illustrates a multi-planar sectional view of the multi-module therapeutic device 10 of FIG. 1A, with the compression plate 22I in a rest position. The medicament reservoir 47 is envisaged to include pre-fed medicament, fed through the filling channel 49. Further, it can be seen in FIG. 3A, that none of the plurality of protrusions 22D of the scroll-wheel design of the third button 22C is in contact with the piston head 22F of the intermediary piston mechanism 22E. As a result, the compression plate 22I seals and prevents any flow of the medicament from the medicament reservoir 47. As the scroll-wheel is rotated any one of the plurality of protrusions 22D may come in contact with the piston head 22F, thereby applying downward pressure on the piston shaft 22G and the compression plate 22I. The downward pressure on the compression plate 22I causes the plurality of compression springs 22H to contract.

FIG. 3B illustrates a multi-planar sectional view of the multi-module therapeutic device 10 of FIG. 1A, with the compression plate 22I in a compressed position. Thus, due to the downward movement of the compression plate 22I, the medicament from the medicament reservoir 47 flows to the plurality of medicament apertures 38A of the compression plate 22I. Moreover, the plurality of medicament apertures 38A of the compression plate 22I, the plurality of medicament channels 38B of the channel plate 22J and the plurality of medicament dispensing apertures 38C of the metal sheet 30, align coaxially with each other allowing the medicament to be dispensed from the metal sheet 30 and onto the skin of the user. The rate of flow and other characteristics of the dispensing of the medicament may be controlled by the processor 52, in correlation with the setting of the medicament flow control interface 60D as discussed in the previously presented discussion in regards to FIG. 2.

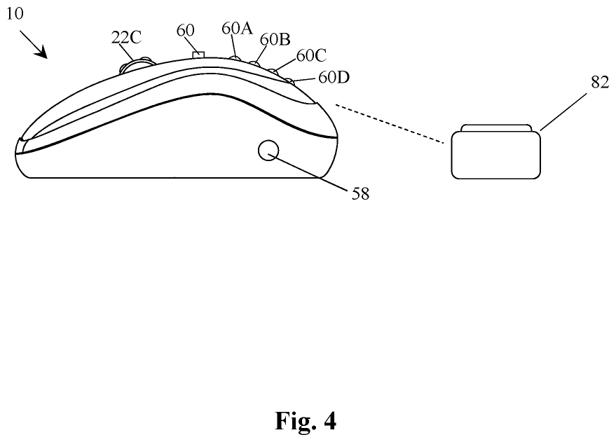

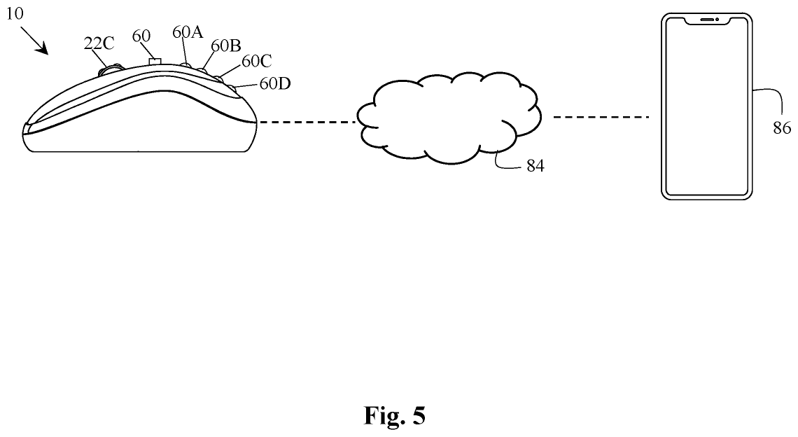

FIG. 4 illustrates the wireless charging of the multi-module therapeutic device 10 of FIG. 1A, in accordance with an embodiment of the present invention. The power supply unit 58 of the device 10 may be charged wireless using a wireless charging unit 82. In that regard, the wireless charging unit 82 may be deploying any one or more of inductive charging, radio charging, charging by power mat, and resonance charging. FIG. 5 illustrates the connection of the multi-module therapeutic device 10 with a remote communication device 86 through a shared network 84, in accordance with an embodiment of the present invention. The connection may be a wired or a wireless connection facilitated by the communication interface 56 of the device 10. The remote communication device 86 may be a smartphone, a tablet computer, a desktop or a notebook, etc. Further, the irradiation control interface 60A, the medicament flow control interface 60D, the vibration control interface 60B, and the temperature control interface 60C are configured to be actuated through the wired or the wireless connection with the remote communication device 86.

While using the device 10, the user may press any one or more of the first button 22A and the second button 22B. The processor 52 executing machine-readable instructions stored in the memory unit 54 would then receive an actuation input from the control panel 20 to actuate one or more of the vibration motor 25 and the plurality of LEDs 40. Additionally, the user may also rotate the scroll-wheel of the third button 22C to actuate the medicament dispenser 48 via the intermediary piston mechanism 22E. Further, the processor 52 would receive one or more of the irradiation control signal from the irradiation control interface 60A, the medicament-flow control signal from the medicament flow control interface 60D, the vibration control signal from the vibration control interface 60B and the temperature control signal from the temperature control interface 60C. Depending upon the actuation of the first 22A, the second 22B and/or the third 22C buttons and the irradiation control signal, the medicament flow control signal, the vibration control signal and/or the temperature control signal, the processor 52 would facilitate the performance of one or more of the following tasks. The tasks would include modifying the emission characteristics of the irradiation emitted by the plurality of LEDs 40, modifying the flow rate of the medicament from the medicament dispenser 48, modifying the vibration characteristics of the vibration of the metal sheet 30 and modifying the temperature of the metal sheet 30.

The present invention provides a personal skincare device that uses several therapeutic modules including a light therapy module, a vibration massage module, a medicament dispensing module, and a thermotherapy module for the treatment of a human skin disorder. Each module is provided with a specific application unit. The personal skincare device of the present invention is implemented in the configuration of a computer mouse while enclosing the aforementioned several therapeutic modules in a single housing. The personal skincare device of the present invention utilizes state of the art LED manufacturing and installation technologies for providing light therapy while also maintaining compactness and economy of the design. The personal skincare device of the present invention includes a metal sheet made of lightweight metal for providing thermotherapy and massage therapy to the skin surface of a user. The device is also configured for wireless charging. Moreover, the functioning of the personal skincare device can be controlled by a remote communication device.

The programming instructions can be, for example, computer-executable and/or logic implemented instructions. In some examples, a computing device is configured to provide various operations, functions, or actions in response to the programming instructions conveyed to the computing device by one or more of the computer-readable medium, the computer recordable medium, and/or the communications medium. The non-transitory computer-readable medium can also be distributed among multiple data storage elements, which could be remotely located from each other. The computing device that executes some or all of the stored instructions can be a micro-fabrication controller or another computing platform. Alternatively, the computing device that executes some or all of the stored instructions could be remotely located computer systems, such as a server.

Further, while one or more operations have been described as being performed by or otherwise related to certain modules, devices or entities, the operations may be performed by or otherwise related to any module, device or entity. As such, any function or operation that has been described as being performed by a module could alternatively be performed by a different server, by the cloud computing platform, or a combination thereof. Further, the operations need not be performed in the disclosed order, although in some examples, an order may be preferred. Also, not all functions need to be performed to achieve the desired advantages of the disclosed system and method, and therefore not all functions are required.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

1. A multi-module therapeutic device for skincare, the multi-module therapeutic device comprising:
a housing, wherein a bottom surface of the housing is planar horizontal in shape;
a plurality of Light Emitting Diodes (LEDs) configured to provide light therapy through emission of electromagnetic radiation;
a vibration motor configured to facilitate a provision of massage therapy;
a metal sheet enclosed within the housing including a plurality of LED apertures for receiving the plurality of LEDs, wherein the metal sheet is configured to act as a medium for application of massage therapy and thermotherapy;
a medicament dispenser configured to dispense a medicament on actuation; and
a control panel provided along a top surface of the housing, wherein the control panel is operably connected with the plurality of LEDs, the vibration motor, and the medicament dispenser.

2. The multi-module therapeutic device as claimed in claim 1, wherein the metal sheet is configured to be heated from heat energy produced by the plurality of LEDs.

3. The multi-module therapeutic device as claimed in claim 1, further comprising an electrical heating element in thermal communication with the metal sheet.

4. The multi-module therapeutic device as claimed in claim 3, wherein the metal sheet is configured to be heated from heat energy received from one or more of the plurality of LEDs and the electrical heating element.

5. The multi-module therapeutic device as claimed in claim 1, wherein the control panel includes at least a first button configured to actuate the plurality of LEDs, a second button configured to actuate the vibration motor, and a third button configured to actuate the medicament dispenser.

6. The multi-module therapeutic device as claimed in claim 1, wherein the housing is in a shape of a computer mouse.

7. The multi-module therapeutic device as claimed in claim 1, wherein the plurality of LEDs are configured to emit the electromagnetic radiation in red and infrared wavelength ranges of the electromagnetic spectrum.

8. The multi-module therapeutic device as claimed in claim 1, wherein the plurality of LEDs have been provided with a plurality of respective auxiliary optics configured to adjust beam angle of the electromagnetic radiation emitted by the plurality of LEDs.

9. The multi-module therapeutic device as claimed in claim 8, wherein the plurality of auxiliary optics includes a plurality of convex lenses.

10. The multi-module therapeutic device as claimed in claim 1, wherein the vibration motor is an eccentric mass motor and is coupled with the metal sheet for providing the massage therapy.

11. The multi-module therapeutic device as claimed in claim 1, wherein the metal sheet is made out of aluminum.

12. The multi-module therapeutic device as claimed in claim 1, wherein the medicament dispenser is configured to be actuated by an intermediary piston mechanism comprising:
a piston head and a piston shaft;
a compression plate provided at a bottom of the piston shaft, wherein the compression plate includes a plurality of medicament apertures; and
a plurality of compression springs provided between a bottom surface of the compression plate and a top surface of a channel plate, wherein a bottom surface of the channel plate includes a plurality of medicament channels that are co-axial with the plurality of medicament apertures.

13. The multi-module therapeutic device as claimed in claim 12, wherein the metal sheet further includes a plurality of medicament dispensing apertures that are coaxial with the plurality of medicament apertures and the plurality of medicament channels.

14. The multi-module therapeutic device as claimed in claim 12, wherein a third button of the control panel is in a form of a scroll-wheel capable of rotating about an axis, wherein the scroll-wheel includes a plurality of protrusions adapted to be in contact with the intermediary piston mechanism on the rotation of the scroll-wheel.

15. The multi-module therapeutic device as claimed in claim 1, wherein the medicament dispenser includes a medicament reservoir.

16. The multi-module therapeutic device as claimed in claim 1, wherein the control panel further includes an irradiation control interface, a medicament flow control interface, a vibration control interface, and a temperature control interface.

17. The multi-module therapeutic device as claimed in claim 16, wherein the irradiation control interface is configured to control emission characteristics of the electromagnetic radiation emitted by the plurality of LEDs, the medicament flow control interface is configured to control a flow rate of the medicament from the medicament dispenser, the vibration control interface is configured to control vibration characteristics of vibration of the metal sheet and the temperature control interface is configured to control a temperature of the metal sheet.

18. The multi-module therapeutic device as claimed in claim 16, wherein the irradiation control interface, the medicament flow control interface, the vibration control interface, and the temperature control interface, are configured to be actuated through a wired or wireless connection with a remote communication device.

19. The multi-module therapeutic device as claimed in claim 16, further comprising a processor, a memory unit and a communication interface, wherein the memory unit includes machine-readable instructions that when executed by the processor, enable the processor to:
receive an actuation input from the control panel to actuate one or more of the vibration motor and the plurality of LEDs;
receive one or more of an irradiation control signal, a medicament flow control signal, a vibration control signal and a temperature control signal; and
perform one or more of modifying characteristics of the electromagnetic radiation emitted by the plurality of LEDs, modifying a flow rate of the medicament from the medicament dispenser, modifying vibration characteristics of the vibration of the metal sheet, and modifying a temperature of the metal sheet.

20. A method for providing multi-module therapy, the method comprising steps of:
providing a multi-module therapeutic device for skincare, the multi-module therapeutic device comprising:
a housing capable of being ergonomically held in one hand, wherein a bottom surface of the housing is planar horizontal in shape,
a plurality of Light Emitting Diodes (LEDs) configured to provide light therapy,
a vibration motor configured to facilitate provision of massage therapy,
a metal sheet enclosed within the housing including a plurality of LED apertures for receiving the plurality of LEDs, wherein the metal sheet is further configured to act as a medium for application of the massage therapy and thermotherapy,
a medicament dispenser, configured to dispense a medicament on actuation, and
a control panel provided along a top surface of the housing, wherein the control panel is operably connected with the plurality of LEDs, the vibration motor, and the medicament dispenser;
receiving an actuation input from the control panel to actuate one or more of the vibration motor and the plurality of LEDs;
receiving one or more of an irradiation control signal, a medicament flow control signal, a vibration control signal and a temperature control signal; and
performing one or more of modifying characteristics of the irradiation emitted by the plurality of LEDs, modifying flow rate of the medicament from the medicament dispenser, modifying the vibration characteristics of the vibration of the metal sheet, and modifying the temperature of the metal sheet.
